(19) United States
(12) Patent Application Publication
(10) Pub. No.: US 2022/0348230 A1
(43) Pub. Date: Nov. 3, 2022

(54) SERVER, SYSTEMS AND METHODS FOR AUTOMATING SAFETY ACTIONS USING MONITORED PARAMETERS

(71) Applicant: Mindset Medical, Inc., Phoenix, AZ (US)

(72) Inventors: Derek Michael Mulgrew, Spokane, WA (US); Mitchell Andrew Foster, Scottsdale, AZ (US); Neil Robert Crawford, Chandler, AZ (US)

(21) Appl. No.: 17/735,401

(22) Filed: May 3, 2022

Related U.S. Application Data
(60) Provisional application No. 63/183,613, filed on May 3, 2021.

(51) Int. Cl. B60W 60/00; A61B 5/00; A61B 5/0205; A61B 5/021; A61B 5/024; A61B 5/145; A61B 5/103; A61B 5/107; A61B 5/11; B60W 50/16; B60W 10/04; B60W 10/20; B60W 50/00

(52) U.S. Cl. CPC B60W 60/0016; A61B 5/0075; A61B 5/0077; A61B 5/02055; A61B 5/021; A61B 5/024; A61B 5/14532; A61B 5/14542; A61B 5/1032; A61B 5/1077; A61B 5/11; A61B 5/7267; A61B 5/6893; B60W 50/16; B60W 10/04; B60W 10/20; B60W 50/0098; A61B 2562/0219; A61B 2562/0271; A61B 2562/0204; B60W 2050/143

(57) ABSTRACT

Systems and methods described herein are directed toward executing an intervention based on monitored physiological and/or environmental parameters according to some embodiments. In some embodiments, the system is configured to execute the intervention if one or more parameters are outside a pre-determined threshold. In some embodiments, an intervention can include and audible message. In some embodiments, the intervention can include taking control of a vehicle. In some embodiments, if there is no response from a user, the system will automatically contact a first responder and/or will automatically route the vehicle to an emergency facility. In some embodiments, the system includes one or more wearable monitoring devices that send signals to the system about physiological and/or environmental parameters. In some embodiments, the system uses artificial intelligence to determine if an abnormal condition exists.

### RELATED APPLICATION

This application claims the benefit and priority of U.S. Provisional Patent Application No. 63/183,613, filed May 3, 2021, entitled “Automated Safety Action from Optically Tracked Video Vitals: For Automotive, General and Commercial Aviation, Security, Police and Military Use,” which is incorporated herein by reference in its entirety.

### BACKGROUND

Technology exists today to equip vehicles with autonomous or semi-autonomous navigation. For example, airplanes have had autopilot capabilities for many years, and many new automobiles have partially or fully self-driving capabilities. However, there will probably continue to be a need for humans to control vehicles, especially during critical phases of navigation such as takeoff and landing of airplanes.

Unfortunately, humans can suffer from health emergencies at unpredictable times. In the case of an aircraft take-off or landing, for example, a pilot experiencing a medical emergency can cost the lives of many passengers. Likewise, in the case of an automobile, a driver losing consciousness can result in the automobile leaving its lane, possibly resulting in a head-on collision with oncoming traffic or hitting pedestrians.

While conventional technology may allow a user to initiate autonomous navigation, there is not currently a way to monitor a person's physiological parameters or environmental conditions to an extent where a computer can make a decision to take control of a vehicle or initiate an emergency response. Therefore, there is a need in the art for systems and methods to initiate automated safety actions from monitored physiological parameters and/or environmental conditions.

### SUMMARY

In some embodiments, systems and methods described herein are directed to technologies configured to monitor physiological parameters such as health vitals through one or more sensors. In some embodiments, as a non-limiting example, one or more sensors include a camera such as the camera on a smartphone and/or another conventional camera. In some embodiments, the system is configured to input one or more images from one or more sensors to continuously or periodically measure heart rate, breathing rate, oxygen saturation, and blood pressure. In some embodiments, another non-limiting example of a sensor includes a wearable device such as an adhesive patch configured to measure temperature or provide an alternate/redundant measurement for one or more other sensors.

In some embodiments, the system includes one or more monitoring sensors, one or more vehicle controllers and one or more computers comprising one or more processors and one or more non-transitory computer readable media, the one or more non-transitory computer readable media including instructions stored thereon that when executed cause the one or more computers to implement one or more steps.

In some embodiments, the one or more steps include receiving, by the one or more processors, one or more monitoring signals from one or more monitoring sensors. In some embodiments, the one or more steps include analyzing, by the one or more processors, the one or more monitoring signals at predetermined intervals. In some embodiments, the one or more steps include determining, by the one or more processors, that the one or more monitoring signals are outside one or more predetermined thresholds. In some embodiments, the one or more steps include executing, by the one or more processors, an intervention when the one or more monitoring signals are outside of the one or more predetermined thresholds.

In some embodiments, the one or more monitoring sensors are configured and arranged to enable the system to monitor and/or analyze one or more physiological parameters of a user's body via the one or more monitoring signals. In some embodiments, the one or more monitoring signals correlate to the one or more physiological parameters. In some embodiments, the intervention includes one or more phases. In some embodiments, first phase of the one or more phases includes a communication with a user. In some embodiments, the communication includes one or more of an audible communication, a visual communication, and physical communication.

In some embodiments, the system is configured to receive a response from the user. In some embodiments, system is configured to initiate one or more second phase control steps if there is no user response and/or an abnormal user response. In some embodiments, one or more control steps include executing, by the one or more processors, control over a vehicle via the one or more vehicle controllers. In some embodiments, the one or more control steps include the system steering the vehicle to a side of a road and bring the vehicle to a safe stop.

In some embodiments, the system is configured to input one or more environmental parameters before implementing the intervention. In some embodiments, one or more environmental parameters include an object's proximity to a user. In some embodiments, the one or more monitoring sensors are configured determine the object's proximity to the user.

In some embodiments, the system includes a global positioning system. In some embodiments, the system is configured to interface with one or more traffic databases. In some embodiments, the system is configured to input speed limit data from the one or more traffic databases based on positions determined by the global positioning system when determining that the one or more monitoring signals are outside the one or more predetermined thresholds.

In some embodiments, the system is configured to execute the intervention even if no abnormal physiological parameters are detected from the one or more monitoring signals. In some embodiments, the system is configured to execute the intervention if one or more vehicle parameters are abnormal. In some embodiments, the one or more control steps include the system autonomously transporting the vehicle and/or driver to an emergency center and/or emergency responder in a third phase.

In some embodiments, the one or more monitoring sensors include cameras (including infrared devices), microphones, accelerometers, heartrate monitors, blood pressure monitors, blood sugar monitors, blood oxygen monitors, pulse monitors, thermometers, weight monitors, and/or electrodes. In some embodiments, the one or more physiological parameters include one or more vital signs, positions, shapes, colors, and/or movements of at least a portion of a user's body.

In some embodiments, the analysis performed by the system includes a use of artificial intelligence (AI). In some embodiments, the artificial intelligence includes one or more programming modules including encoder modules, decoder modules, and classifier modules. In some embodiments, the system is configured to input the one or more monitoring signals as training data to train the artificial intelligence. In some embodiments, the artificial intelligence is configured to enable the system to identify specific physiological parameters for a specific user. In some embodiments, the artificial intelligence is configured to enable the system to determine if a physiological parameter is abnormal based on the specific physiological parameters. In some embodiments, the system is configured to accept user feedback for training the AI and/or before executing an intervention.

### DRAWING DESCRIPTION

FIG. 1 illustrates a computer system 1010 enabling or comprising the systems and methods in accordance with some embodiments of the system.

### DETAILED DESCRIPTION

The following non-limiting examples of some embodiments of the system implemented in vehicles are meant to aid those of ordinary skill in understanding the scope of the disclosure. Although some embodiments may be directed to an aircraft, some other embodiments may be directed to an automobile, and still other embodiments directed to wearable configurations, the system and methods described in relation to each are interchangeable and are not to be interpreted as being confined to their example implementation.

In some embodiments, the system includes an automotive monitoring and recovery system (hereafter referred to as the “recovery system”). In some embodiments, the system includes one or more monitoring sensors. In some embodiments, one or more monitoring sensors include cameras (including infrared), microphones, accelerometers, heartrate monitors, blood pressure monitors, blood sugar monitors, blood oxygen monitors, pulse monitors, thermometers, weight monitors, electrodes, and the like. In some embodiments, the system is configured to receive one or more monitoring signals from one or more monitoring sensors. In some embodiments, the system includes a remote computer configured to receive, record, and/or send the monitoring signals on a predetermined interval and/or a continuous basis. In some embodiments, the system includes an application program (App) configured to send and/or receive one or more monitoring signals from the remote computer (e.g., a cellular phone). In some embodiments, one or more monitoring sensors are conventional monitoring sensors implemented in the system in a novel way further described herein.

In some embodiments, the one or more monitoring sensors are configured to enable a computer system to monitor and/or analyze one or more portions of a user's (e.g., driver, pilot) body. In some embodiments, one or more portions of a driver's body include one or more portions of the head including eyes, ears nose, mouth, cheeks, hair, and neck. In some embodiments, one or more portions of a driver's body include one or more shoulders, arms, hands, fingers, chest, stomach, legs, and feet. In some embodiments, the system is configured to analyze one or more physiological parameters of a driver's body including one or more vital signs, positions, shapes, colors, and/or movements of at least a portion of a driver's body. In some embodiments, the system is configured to implement one or more actions described herein upon determining an abnormal condition has occurred to one or more portions of a driver's body.

In some embodiments, the system includes artificial intelligence (AI) configured to determine a change in a user's physiological parameters. In some embodiments, the AI includes one or more programming structures including encoders, decoders, and classifier modules configured to interface with one or more system features described herein. In some embodiments, the system is configured to record one or more normal physiological parameters during normal operation. In some embodiments, the system is configured to input the one or more normal physiological parameters as a training set to train the AI to identify abnormal physiological parameters for one or more users.

As a non-limiting example, if a user typically does not node their head repeatably while driving and begins to do so while the vehicle is being operated, one or more system actions described herein can be implemented according to some embodiments. Another non-limiting example is a user who typically keeps both hands on the steering wheel, where the system determines an abnormal physiological parameter when the user continuously places one hand on their chest and/or or squeezes their eyes repeatably. In some embodiments, the system is configured to present the user with options (e.g., audible prompt or other prompts) for feedback as to whether to include a certain type of behavior as normal. In some embodiments, the system is configured to record a user's training data and/or AI configurations on one or more non-transitory computer readable media, and enable uploading (e.g., through a wired and/or wireless network connection) to various system hardware and/or software components in different vehicles. In some embodiments, this allows the system to become partially or fully customized or even user specific.

In some embodiments, the one or more monitoring sensors include one or more cameras each configured to attach to one or more camera mounts within an interior of a vehicle. In some embodiments, one or more mounts include, as non-limiting examples, a dashboard mount, a visor mount, a mirror mount, a steering wheel mount, a window mount, and/or any conventional camera mount. In some embodiments, the system includes a steering wheel comprising one or more integrated cameras. In some embodiments, the one or more integrated cameras are detachable from the steering wheel. In some embodiments, the system includes one or more cameras built into a mirror (e.g., rearview mirror) interior. In some embodiments, the mirror glass or other displays include one-way glass configured to enable a camera to monitor a driver through the one-way glass.

In some embodiments, the one or more monitoring sensors are configured and arranged to monitor one or more users' one or more physiological parameters and input monitored signals to the system to determine if the one or more physiological parameters are abnormal and/or unsafe. In some embodiments, one or more physiological parameters include breathing rate (e.g., too fast or too slow), heart rate (e.g., too fast or too slow), and/or blood pressure (e.g., too high or too low). In some embodiments, one or more physiological parameters include eyes closed or rolled for a prolonged period which may indicate loss of consciousness or seizure. In some embodiments, one or more physiological parameters include pupil dilation inconsistent with ambient lighting and/or AI training sets which may indicate a neurological problem. In some embodiments, one or more physiological parameters include lowered oxygen saturation which may indicate hypoxia. In some embodiments, one or more physiological parameters include blood alcohol level which may indicate intoxication. In some embodiments, one or more physiological parameters include body temperature and/or environmental temperature, which could indicate a fever or environmental hazard, respectively. In some embodiments, one or more physiological parameters include lowered blood glucose which could indicate a diabetic event.

In some embodiments, the system is configured to input monitoring data (e.g., signals) received from one or more monitoring sensors and analyze the monitor signals at predetermined intervals (e.g., between 0.1 second to 1 second) and make decisions about intervention when one or more physiological parameters are outside of acceptable thresholds. In some embodiments, the system is configured to output non-emergency status updates to the user periodically (e.g., 15 to 60 minute intervals). A non-limiting example of a status update includes an audio message “your heart rate is 10% slower and your eyes are closing for 20% longer than at the beginning of the trip.”

In some embodiments, the system is configured to implement an intervention upon determining one or more abnormal physiological parameters and/or environmental parameters are an indication of a hazardous health condition. In some embodiments, an intervention includes one or more phases.

In some embodiments, the first phase is an initial intervention phase. In some embodiments, a first phase include communication with the driver. In some embodiments, the communication includes one or more of an audible, visual, and physical communication. In some embodiments, an audible communication non-limiting example may include the system initiating communication (e.g., a report on changes of one or more physiological parameters) through one or more speakers. In some embodiments, a visual communication non-limiting example may include the system initiating communication (e.g., warning light, text) on a display such as a dashboard and/or display screen. In some embodiments, a physical communication non-limiting example may include the system initiating communication via one or more motors configured to create a vibration in the seat or in a steering wheel.

In some embodiments, the system is configured to accept user feedback input that the warning is erroneous. In some embodiments, the system is configured to use the feedback input to train the AI. In some embodiments, the system is configured to initiate one or more other phases (e.g., a second phase) if there is no user response and/or an abnormal user response. In some embodiments, the feedback input can include an audible response from the user input to one or more speakers, and/or can include a physical input including the actuation of one or more actuators (e.g., buttons, switches) as non-limiting examples.

In some embodiments, one or more phases include a second phase. In some embodiments, the second phase is a mild intervention phase. In some embodiments, the system is configured to initiate one or more control steps of the second phase if there is no user response and/or an abnormal user response. In some embodiments, the system includes a vehicle control system. In some embodiments, the vehicle control system is configured to control the vehicle autonomously and/or semi-autonomously. In some embodiments, the vehicle control system includes one or more cameras, motors, actuators, global positioning systems (GPS) and/or sensors configured to enable the system to initiate one or more intervention programs. In some embodiments, the system is configured to interface with one or more conventional vehicle control systems to initiate one or more intervention programs.

In some embodiments, one or more control steps include the system overriding user control of the vehicle. In some embodiments, one or more control steps include the system steering and/or changing the velocity of the vehicle. one or more control steps include the system initiating one or more vehicle emergency systems. In some embodiments, a non-limiting example of one or more control steps include the system steering the car to the side of the road and bringing the vehicle to a safe stop. In some embodiments, a non-limiting example of one or more control steps include the system automatically engaging the hazard lights. In some embodiments, one or more control steps include the system initiating one or more emergency communications with the driver. In some embodiments, one or more emergency communications include, as a non-limiting example, an audible message through one or more speakers warning the user that further action will be taken if there is no response and/or an abnormal response. In some embodiments, the system is configured to implement one or more other phases (e.g., a third phase) and/or one or more other control steps if there is no response and/or an abnormal response from the driver.

In some embodiments, one or more phases include a third phase. In some embodiments, the third phase is a strong intervention phase. In some embodiments, one or more steps in a third phase include contacting an emergency responder. In some embodiments, one or more control steps of the third phase include the system locating the nearest emergency center (e.g., hospital, emergency care, fire station) and/or an emergency responder. In some embodiments, one or more control steps in a third phase include the system autonomously transporting the vehicle and/or driver to an emergency center and/or emergency responder.

In some embodiments, the system is configured to implement one or more steps of one or more phases even if no abnormal physiological parameters are detected. In some embodiments, the system is configured to implement one or more steps of one or more phases if one or more vehicle parameters are abnormal. A non-limiting example of abnormal vehicle parameters include the vehicle velocity changing more than a predetermined value (e.g., percentage, setpoint, or other values).

In some embodiments, the system is configured to input one or more environmental factors during an analysis of an abnormal vehicle parameter. In some embodiments, one or more environmental factors include a speed limit. In some embodiments, the system is configured to access one or more traffic databases to obtain a speed limit at a GPS location. In some embodiments, the system is configured to access one or more third party traffic databases (e.g., Google Maps® or other available databases.)

In some embodiments, one or more environmental factors include weather conditions. In some embodiments, the system is configured to access one or more weather databases to determine weather conditions in the area. In some embodiments, the system is configured to determine weather conditions from one or more sensors (e.g., cameras, rain sensors, traction control sensors). In some embodiments, the system is configured to determine if a change in vehicle velocity was normal based on an analysis of one or more environmental factors.

In some embodiments, one or more environmental factors include traffic conditions. In some embodiments, the system includes one or more sensors configured to recognize physical objects (e.g., cars, bikes, people) within a proximity of the vehicle. In some embodiments, the system is configured to analyze a change in velocity and/or position of one or more proximate physical objects to determine if the change in velocity and/or position is the cause of the vehicle change in velocity (speed and/or direction). In some embodiments, the system is configured to implement and/or not implement one or more steps of one or more phases based on the analysis.

In some embodiments, one or more environmental factors include traffic guides (e.g., lights, signs, lines, reflectors, markers, electronic signals, etc.). In some embodiments, the system includes one or more sensors configured to recognize traffic guides. In some embodiments, the system is configured to analyze a state of one or more traffic guides to determine if the change in vehicle velocity was the result of a state of one or more traffic guides. In some embodiments, the system is configured to implement and/or not implement one or more steps of one or more phases based on the analysis. In some embodiments, the system is configured to skip one or more phases and/or one or more steps if it is determined that immediate action must be taken where autonomous control by the system is needed to avoid danger (e.g., an accident, injury or death).

As a non-limiting example, if the system determines that the vehicle should be moving because there is no object in front of it and a traffic light is green, the system is configured to initiate one or more steps of one or more phases described herein according to some embodiments. Another non-limiting example includes the system determining that the vehicle is excessively and/or erratically moving between two painted road lines, and initiating one or more steps of one or more phases described herein according to some embodiments. Another non-limiting example may include input from a weight sensor in the seat indicating that the driver is moving erratically (e.g., convulsing), wherein the system skips the first phase and/or the second phase intervention and implements a strong intervention normally reserved for a third phase according to some embodiments. Those of ordinary skill would understand that the system can be configured to skip and/or implement any intervention phase and/or step in any order and still fall within the scope of the disclosure according to some embodiments.

As described above, in some embodiments, one or more aspects of the system are configured to be implemented on any type of vehicle including cars, boats, aircraft, trains, and/or any object configured to transport a user and/or where the user has at least partial control over the vehicle. However, the system is not limited to mounted sensors in some embodiment. In some embodiments, the system includes one or more monitoring sensors configured to be coupled to (e.g., worn by) a user. In some embodiments, the system comprises one or more worn monitoring sensors and/or one or more worn monitoring sensors. Some non-limiting examples of worn monitor sensors include an eyewear mounted and/or an integrated camera(s) (including night vision and/or thermal imaging), garments (including handcuffs and/or restraints) with electrodes, garments with conventional medical equipment monitoring, a body camera(s) configured to attach to a vest or belt, and the like.

In some embodiments, the one or more worn monitoring sensors are configured to monitor a user's physiological parameters and/or a subject's physiological parameters. In some embodiments, non-limiting examples of a user may include a law enforcement officer or military personnel (although the system is compatible with any living creature or robot), where a non-limiting example of a subject by include an individual proximate to the user, including a suspect or perpetrator.

In some embodiments, the system is configured to perform any of the afore mentioned phases and/or steps by inputting the data received from one or more worn monitoring sensors for analysis. In addition, in some embodiments, the system is configured to perform any of the afore mentioned phases and/or steps by inputting the data received about a subject from one or more worn monitoring sensors for analysis. In some embodiments, a non-limiting example of the system receiving subject data includes the system determining that a subject's breathing rate is abnormal (e.g., heavy or shallow breathing). Another non-limiting example according to some embodiments is elevated heart rate and/or blood pressure received from one or more heart rate sensors integrated into handcuffs. A body camera transmitting images to the system where the system alerts the user (audibly and/or visually) that the system has determined that a subject's closed and/or rolled eyes is indicative of intoxication and/or a seizure is another non-limiting example according to some embodiments.

Still further, non-limiting examples according to some embodiments include: pupil dilation inconsistent with ambient light, where the system determines that the subject is suffering from a neurological event and/or under the influence of drugs from images received from one or more cameras; oxygen sensors within a user's glove sending signals enabling the system to determine the user is experiencing hypoxia; an odor sensor attached to a vest configured to detect and transmit signals indicating the presence of alcohol in the environment; a wireless receiver configured to receive a signal from a subject's glucose monitor when the user is within a predetermined proximity; and a thermal measuring device (e.g., thermal sensor, laser sensor, thermal imaging device, etc.) configured to transmit a user's temperature for system analysis such as a change in temperature of at least a portion of a subject's body (e.g., face, hands). In some embodiments, any type of worn monitoring sensor associated with a particular garment can be readily exchangeable and/or incorporable with any other type of garment and/or one or more other worn monitoring sensors.

In some embodiments, the system is configured to initiate one or more intervention phases including one or more intervention steps. In some embodiments, one or more intervention phases have been previously described. In some embodiments, a first intervention phase includes an audible and/or visual signal of an abnormal physiological parameter. In some embodiments, a non-limiting example of an audible signal includes a system defined message played through one or more worn speakers, such as speakers integrated into eyewear. In some embodiments, a non-limiting example of a visual signal includes a message displayed on augmented reality eyewear.

In some embodiments, a second phase includes automatically calling for backup and/or one or more emergency responders. In some embodiments, the system includes a worn GPS (e.g., in a watch or cellphone) and is configured to send a user's location and/or any information received by one or more monitoring sensors to one or more emergency systems and/or personnel. In some embodiments, the system is configured to alert the user to the type of medical emergency is occurring based on the analysis. In some embodiments, a type of medical emergency output by the system may include possible drugs the person has consumed. As a non-limiting example, increased and/or irregular heartrate received as one or more inputs can result in methamphetamine as one of one or more possible diagnosis. In some embodiments, the system is configured to communicate what diagnosis are unlikely (e.g., by percentage and/or statistical confidence level). In some embodiments, the system is configured to respond to one or more commands (e.g., voice commands) requesting confirmation that a diagnosis is accurate. In some embodiments, the system is configured to access one or more databases comprising emergency response instructions and provide the emergency response instructions to the user. In some embodiments, the system is configured to monitor the user and/or the subject and analyze the implementation of the instructions by the user and provide feedback to the user on further steps or correct steps. In some embodiments, the system is configured to initiate communication between the user and a medical professional to enable the medical professional to provide instructions to the user. In some embodiments, the system is configured to enable the medical professional to access one or more system received inputs from one or more monitoring sensors and/or one or more system analysis results. In some embodiments, such analysis, instruction, and/or communication can help guide the user to respond with appropriate dialog and/or force and/or techniques.

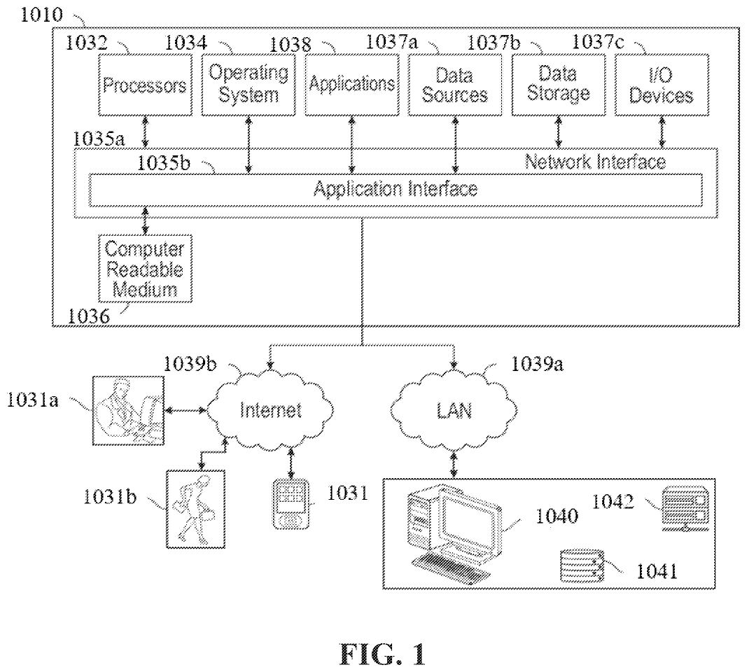

FIG. 1 illustrates a computer system 1010 enabling or comprising the systems and methods in accordance with some embodiments of the system. In some embodiments, the computer system 1010 can operate and/or process computer-executable code of one or more software modules of the aforementioned system and method. Further, in some embodiments, the computer system 1010 can operate and/or display information within one or more graphical user interfaces (e.g., HMIs) integrated with or coupled to the system.

In some embodiments, the computer system 1010 can comprise one or more processors 1032. In some embodiments, the one or more processors 1032 can reside in, or coupled to, one or more conventional server platforms (not shown). In some embodiments, the computer system 1010 can include a network interface 1035a and an application interface 1035b coupled to the least one processor 1032 capable of processing at least one operating system 1034. Further, in some embodiments, the interfaces 1035a, 1035b coupled to at least one processor 1032 can be configured to process one or more of the software modules (e.g., such as enterprise applications 1038). In some embodiments, the software application modules 1038 can include server-based software and can operate to host at least one user account and/or at least one client account, and operate to transfer data between one or more of these accounts using the at least one processor 1032.

With the above embodiments in mind, it is understood that the system can employ various computer-implemented operations involving data stored in computer systems. Moreover, the above-described databases and models described throughout this disclosure can store analytical models and other data on computer-readable storage media within the computer system 1010 and on one or more non-transitory computer-readable storage media coupled to the computer system 1010 according to various embodiments. In addition, in some embodiments, the above-described applications of the system can be stored on computer-readable storage media within the computer system 1010 and on computer-readable storage media coupled to the computer system 1010. In some embodiments, these operations are those requiring physical manipulation of physical quantities. Usually, though not necessarily, in some embodiments these quantities take the form of one or more of electrical, electromagnetic, magnetic, optical, or magneto-optical signals capable of being stored, transferred, combined, compared and otherwise manipulated. In some embodiments, the computer system 1010 can comprise at least one computer readable medium 1036 coupled to at least one of at least one data source 1037a, at least one data storage 1037b, and/or at least one input/output 1037c. In some embodiments, the computer system 1010 can be embodied as computer readable code on a computer readable medium 1036. In some embodiments, the computer readable medium 1036 can be any data storage that can store data, which can thereafter be read by a computer (such as computer 1040). In some embodiments, the one or more non-transitory computer readable medium 1036 can be any physical or material medium that can be used to tangibly store the desired information or data or instructions and which can be accessed by a computer 1040 or processor 1032. In some embodiments, the computer readable medium 1036 can include hard drives, network attached storage (NAS), read-only memory, random-access memory, FLASH based memory, CD-ROMs, CD-Rs, CD-RWs, DVDs, magnetic tapes, other optical and non-optical data storage. In some embodiments, various other forms of computer-readable media 1036 can transmit or carry instructions to a remote computer 1040 and/or at least one user 1031, including a router, private or public network, or other transmission or channel, both wired and wireless. In some embodiments, the software application modules 1038 can be configured to send and receive data from a database (e.g., from a computer readable medium 1036 including data sources 1037a and data storage 1037b that can comprise a database), and data can be received by the software application modules 1038 from at least one other source. In some embodiments, at least one of the software application modules 1038 can be configured within the computer system 1010 to output data to at least one user 1031 via at least one graphical user interface rendered on at least one digital display.

In some embodiments, the one or more non-transitory computer readable media 1036 can be distributed over a conventional computer network via the network interface 1035a where the system embodied by the computer readable code can be stored and executed in a distributed fashion. For example, in some embodiments, one or more components of the computer system 1010 can be coupled to send and/or receive data through a local area network (“LAN”) 1039a and/or an internet coupled network 1039b (e.g., such as a wireless internet). In some embodiments, the networks 1039a, 1039b can include wide area networks (“WAN”), direct connections (e.g., through a universal serial bus port), or other forms of computer-readable media 1036, or any combination thereof.

In some embodiments, components of the networks 1039a, 1039b can include any number of personal computers 1040 which include for example desktop computers, and/or laptop computers, or any fixed, generally non-mobile internet appliances coupled through the LAN 1039a. For example, some embodiments include one or more of personal computers 1040, databases 1041, and/or servers 1042 coupled through the LAN 1039a that can be configured for any type of user including an administrator. Some embodiments can include one or more personal computers 1040 coupled through network 1039b. In some embodiments, one or more components of the computer system 1010 can be coupled to send or receive data through an internet network (e.g., such as network 1039b). For example, some embodiments include at least one user 1031a, 1031b, is coupled wirelessly and accessing one or more software modules of the system including at least one enterprise application 1038 via an input and output (“I/O”) 1037c. In some embodiments, the computer system 1010 can enable at least one user 1031a, 1031b, to be coupled to access enterprise applications 1038 via an I/O 1037c through LAN 1039a. In some embodiments, the user 1031 can comprise a user 1031a coupled to the computer system 1010 using a desktop computer, and/or laptop computers, or any fixed, generally non-mobile internet appliances coupled through the internet 1039b. In some embodiments, the user can comprise a mobile user 1031b coupled to the computer system 1010. In some embodiments, the user 1031b can connect using any mobile computing 1031c to wireless coupled to the computer system 1010, including, but not limited to, one or more personal digital assistants, at least one cellular phone, at least one mobile phone, at least one smart phone, at least one pager, at least one digital tablets, and/or at least one fixed or mobile internet appliances.

The subject matter described herein are directed to technological improvements to the field of ** by **. The disclosure describes the specifics of how a machine including one or more computers comprising one or more processors and one or more non-transitory computer readable media implement the system and its improvements over the prior art. The instructions executed by the machine cannot be performed in the human mind or derived by a human using a pen and paper but require the machine to convert process input data to useful output data. Moreover, the claims presented herein do not attempt to tie-up a judicial exception with known conventional steps implemented by a general-purpose computer; nor do they attempt to tie-up a judicial exception by simply linking it to a technological field. Indeed, the systems and methods described herein were unknown and/or not present in the public domain at the time of filing, and they provide technologic improvements advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Any text in the drawings is part of the system's disclosure and is understood to be readily incorporable into any description of the metes and bounds of the system. Any functional language in the drawings is a reference to the system being configured to perform the recited function, and structures shown or described in the drawings are to be considered as the system comprising the structures recited therein. Any figure depicting a graphical user interface is a disclosure of the system configured to display the contents of the graphical user interface. It is understood that defining the metes and bounds of the system using a description of images in the drawing does not need a corresponding text description in the written specification to fall with the scope of the disclosure.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system. The phrase “configured to” also denotes the step of configuring a structure or computer to execute a function in some embodiments.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with operatively connected when used in conjunction with physical structures.

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The previous detailed description is to be read with reference to the figures, in which like elements in different figures have like reference numerals. The figures, which are not necessarily to scale, depict some embodiments and are not intended to limit the scope of embodiments of the system.

Any of the operations described herein that form part of the invention are useful machine operations. The invention also relates to a device or an apparatus for performing these operations. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g. a cloud of computing resources.

The embodiments of the invention can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

We claim:

1. A system for initiating responses to emergency situations comprising:
one or more monitoring sensors;
one or more vehicle controllers;
one or more computers comprising one or more processors and one or more non-transitory computer readable media, the one or more non-transitory computer readable media including instructions stored thereon that when executed cause the one or more computers to implement:
receive, by the one or more processors, one or more monitoring signals from one or more monitoring sensors;
analyze, by the one or more processors, the one or more monitoring signals at predetermined intervals;
determine, by the one or more processors, that the one or more monitoring signals are outside one or more predetermined thresholds; and
execute, by the one or more processors, an intervention when the one or more monitoring signals are outside of the one or more predetermined thresholds;
wherein the one or more monitoring sensors are configured and arranged to enable the system to monitor and/or analyze one or more physiological parameters of a user's body via the one or more monitoring signals; and
wherein the one or more monitoring signals correlate to the one or more physiological parameters.

2. The system of claim 1,
wherein the intervention includes one or more phases;
wherein a first phase of the one or more phases includes a communication with a user; and
wherein the communication includes one or more of an audible communication, a visual communication, and physical communication.

3. The system of claim 2,
wherein the system is configured to receive a response from the user;
the system is configured to initiate one or more second phase control steps if there is no user response and/or an abnormal user response.

4. The system of claim 3,
wherein one or more control steps include executing, by the one or more processors, control over a vehicle via the one or more vehicle controllers.

5. The system of claim 4,
wherein the one or more control steps include the system steering the vehicle to a side of a road and bring the vehicle to a safe stop.

6. The system of claim 1,
wherein the system is configured to input one or more environmental parameters before implementing the intervention.

7. The system of claim 6,
wherein the one or more environmental parameters include an object's proximity to a user; and
wherein the one or more monitoring sensors are configured determine the object's proximity to the user.

8. The system of claim 6,
wherein the system includes a global positioning system;
wherein the system is configured to interface with one or more traffic databases; and
wherein the system is configured to input speed limit data from the one or more traffic databases based on a position determined by the global positioning system when determining that the one or more monitoring signals are outside the one or more predetermined thresholds.

9. The system of claim 1,
wherein the system is configured to execute the intervention even if no abnormal physiological parameters are detected from the one or more monitoring signals; and
wherein the system is configured to execute the intervention if one or more vehicle parameters are abnormal.

10. The system of claim 4,
wherein the one or more control steps include the system autonomously transporting the vehicle and/or driver to an emergency center and/or emergency responder in a third phase.

11. The system of claim 1,
wherein the one or more monitoring sensors include cameras, thermal imaging devices, microphones, accelerometers, heartrate monitors, blood pressure monitors, blood sugar monitors, blood oxygen monitors, pulse monitors, thermometers, weight monitors, and/or electrodes.

12. The system of claim 1,
wherein the one or more physiological parameters include one or more vital signs, positions, shapes, colors, and/or movements of at least a portion of a user's body;

13. The system of claim 1,
wherein the analysis includes a use of artificial intelligence;
wherein the artificial intelligence includes one or more programming modules including encoder modules, decoder modules, and classifier modules; and
wherein the system is configured to input the one or more monitoring signals as training data to train the artificial intelligence.

14. The system of claim 13,
wherein the artificial intelligence is configured to enable the system to identify specific physiological parameters for a specific user; and
wherein the artificial intelligence is configured to enable the system to determine if a physiological parameter is abnormal based on the specific physiological parameters.

15. The system of claim 1,
wherein the system is configured to accept user feedback before executing the intervention.
